(19) United States
(12) Patent Application Publication
(10) Pub. No.: US 2007/0299330 A1
(43) Pub. Date: Dec. 27, 2007

(54) SENSOR, PROCESSING MEANS, METHOD AND COMPUTER PROGRAM FOR PROVIDING INFORMATION ON A VITAL PARAMETER OF A LIVING BEING

(76) Inventors: Robert Couronne, Erlangen (DE); Stephan Bode, Mannheim (DE); Stefan Aschenbrenner, Eckental (DE); Hans-Joachim Moersdorf, Furth (DE)

Correspondence Address: GLENN PATENT GROUP, 3475 EDISON WAY, SUITE L, MENLO PARK, CA, 94025, US

(21) Appl. No.: 11/749,668

(22) Filed: May 16, 2007

(30) Foreign Application Priority Data
May 24, 2006 (DE) 102006024459.1

(51) Int. Cl. A61B 5/02

(52) U.S. Cl. 600/368; 600/300; 600/500

(57) ABSTRACT

A sensor for providing information on a vital parameter includes a mounter for attaching the sensor to a living being, a light source connected to the mounter to radiate light into a part of the body of the living being, and a light receiver connected to the mounter and implemented to receive part of the light to provide, in dependence on an intensity of the light received, a light intensity signal depending on the vital parameter. Additionally, the sensor includes an acceleration sensor connected to the mounter and implemented to provide an acceleration signal in dependence on an acceleration in at least one direction. The sensor is implemented to transfer the light intensity signal and the acceleration signal to a processor for a combining processing of the light intensity signal and the acceleration signal, or to generate the light intensity signal in dependence on the acceleration signal.

### CROSS-REFERENCE TO RELATED APPLICATIONS

This application claims priority from German Patent Application No. 10 2006 024 459.1, which was filed on May 24, 2006, and is incorporated herein by reference in its entirety.

### TECHNICAL FIELD

The present invention generally relates to a sensor, processing means and a computer program for providing information on a vital parameter of a living being, in particular to transmission plethysmography sensitive to movement artifacts performed at the wrist.

### BACKGROUND

In the field of medicine, it is necessary in many situations to detect vital parameters of a human being and/or living being. Further, it has shown that it is desirable with an increasing automation to be able to detect the vital parameters continually in an electronical form. A way of determining important vital parameters of a human being and/or a living being is recording a plethysmogram. A plethysmogram is a graphical representation of volume changes. In medicine, a plethysmogram is, among other things, used to represent volume changes of arterial blood vessels in the human body. For recording a plethysmogram at a patient, a sensor device is typically used which contains a light source and a photoreceiver and which is such that light passes the tissue layers and the remaining light intensity is measured by the photoreceiver. When the light passes the tissue layers, it is subjected to attenuation which is dependent on, among other things, the wavelength of the light, the type and concentration of the components in the tissue irradiated and volume changes of the arterial blood flow. The photoreceiver transforms the incident light to a photocurrent the amplitude of which is modulated by volume changes of the arterial blood vessels caused by contractions of the cardiac muscle.

Known plethysmographs are usually applied to the finger or earlobe of the patient since the top skin layers there are interspersed very densely by arterial blood vessels and since in addition the attenuating influence of bones or adipose tissue is minimal. Plethysmographs based on both the transmission principle and the remission principle are used here.

In the remission method, the finger is not radiated through completely, as is the case in the transmission method, but the light portion emitted by the tissue after the light irradiation is measured.

All photoplethysmographs for being employed at the finger, exemplarily mounted to the fingertip by a fingerclip, share a limitation in the patient's freedom to move. In addition, it has been found out that conventional plethysmographs provide unreliable values under some operating conditions.

### SUMMARY

According to an embodiment, a sensor for providing information on a vital parameter of a living being may have mounting means for attaching the sensor to the living being, a light source connected to the mounting means to radiate light into a part of the body of the living being, a light receiver connected to the mounting means and implemented to receive part of the radiated light to provide, depending on an intensity of the light received, a light intensity signal depending on the vital parameter, and an acceleration sensor connected to the mounting means and implemented to provide, in dependence on an acceleration in at least one direction, an acceleration signal, wherein the sensor is implemented to transfer the light intensity signal and the acceleration signal to processing means for a combining processing of the light intensity signal and the acceleration signal or to generate the light intensity signal in dependence on the acceleration signal.

According to another embodiment, a processing means for providing information on a vital parameter of a living being based on a light intensity signal and an acceleration signal from a sensor, the light intensity signal describing an intensity of light received from a light receiver attached to a living being from a part of the body of the living being, and the acceleration signal describing an acceleration at the location of an acceleration sensor mechanically connected to the light receiver, may have: means implemented to combine the light intensity signal and the acceleration signal to determine the information on the vital parameter.

According to another embodiment, a method for providing information on a vital parameter of a living being, may have the steps of: determining information on an optical attenuation in a part of the body of the living being between a light source and a light receiver, wherein the optical attenuation depends on the vital parameter, and wherein the light source and the light receiver are attached to the part of the body by mounting means; determining information on an acceleration of the light source, the light receiver or the mounting means; and combining the information on the optical attenuation and the information on the acceleration to obtain the information on the vital parameter.

According to another embodiment, a method for providing information on a vital parameter of a living being in connection with means having a light source and a light receiver which are arranged to determine information on an optical attenuation in a part of the body of the living being between the light transmitter and the light receiver, the optical attenuation depending on the vital parameter, and the light source and the light receiver being attached to the part of the body by mounting means, may have the steps of: determining information on an acceleration of the light source, the light receiver or the mounting means; and should the information on the acceleration indicate that the acceleration is greater than a predetermined maximally allowed acceleration, switching off the light source and/or interrupting a generation of the information on the vital parameter using the information on the optical attenuation; otherwise determining the information on the vital parameter of the living being from the information on the optical attenuation in the part of the body of the living being between the light source and the light receiver.

An embodiment may have a computer program for performing the methods for providing information on a vital parameter of a living being mentioned before when the computer program runs on a computer.

The central idea of embodiments of the present invention is that the reliability of a sensor for providing information on a vital parameter which evaluates a light intensity transferred by a part of the body of a living being can be improved by evaluating an acceleration of the sensor assembly by an acceleration sensor. It has been found out that the light intensity signal provided by the light receiver is subject to strong variations when an acceleration acts on the sensor assembly. Such an acceleration typically has the result that a relative position between the light source, the part of the body and the light receiver changes and that in addition changes influencing the light intensity signal result due to the acceleration, also within the living being.

It has turned out to be of advantage for the sensor to transfer the light intensity signal and the acceleration signal together to processing means for a combining processing of the light intensity signal and the acceleration signal. By combining the light intensity signal and the acceleration signal, it can be achieved, in the processing means, that errors due to acceleration in the light intensity signal can, for example, be corrected when determining the vital parameter or that the light intensity signal will not be used for calculating the vital parameter should the acceleration sensor determine an acceleration outside an allowed region. Alternatively, reliability information depending on the acceleration signal may be associated to the light intensity signal via the combining processing, the reliability information exemplarily indicating high reliability of the light intensity signal with low an acceleration and vice versa.

Alternatively, it has turned out to be of advantage for the light intensity signal to be generated in dependence on the acceleration signal, i.e. exemplarily, with an acceleration outside an allowed region, generating either no light intensity signal at all (exemplarily by switching off the light source) or generating a corrected light intensity signal.

In other words, the central idea of embodiments of the present invention in the sensor for providing the information on the vital parameter is increasing the reliability of information on the vital parameter established either by providing together the light intensity signal and the acceleration signal for a combining processing, or alternatively determining the light intensity signal in dependence on the acceleration signal and thus taking into consideration the influence of the acceleration on the light intensity signal.

Thus, an embodiment of the present invention provides a sensor for providing information on a vital parameter reacting less sensitive to vibration than conventional sensors and making reliable recording and evaluation of a plethysmogram considerably easier.

In addition, embodiments of the present invention allow increasing a freedom to move for a human being or living being to which the plethysmograph is mounted, in contrast to conventional assemblies. Whereas in conventional plethysmographs the patients and/or human beings or living beings had to be urged not to move and/or only minimally move the part of the body to which the plethysmographs has been mounted, the freedom to move for a human being does not have to be limited considerably by an inventive sensor. By using an acceleration sensor and by the opportunity of a combining processing of the acceleration signal and the light intensity signal, interferences of the light intensity signal due to movements can either be corrected or at least recognized. Exemplarily, it becomes possible to record a plethysmogram at the wrist of a human being without considerably limiting the freedom to move for the human being, wherein nevertheless a reliable information on the vital parameter of the human being is obtained. By recognizing movements, this is possible even under very complicated conditions and/or measuring conditions, as are, for example, present at the wrist. In other words, the conventionally interfering effect occurring due to bones at the wrist moveable to one another being present can basically be compensated by recognizing movements and/or accelerations.

All in all, embodiments of the present invention allow deriving information on a vital parameter from light transfer and/or from optical attenuation between a light source and a light receiver with high reliability, even under complicated conditions, such as, for example, with large movements or accelerations and even when there are bones which are in relative movement to one another when moved.

In an embodiment, the vital parameter includes a pulse frequency of the living being. Here, the light source and the light receiver are arranged such that light transmission and/or optical attenuation in the part of the body between the light source and the light receiver is influenced by a change in the volume of a blood vessel in the part of the body. The dependence of the optical attenuation in the part of the body on movements and/or accelerations in turn can be compensated by combining the light intensity signal and the acceleration signal, or it can at least be recognized when the light intensity signal is unreliable due to great movements and/or accelerations.

In another embodiment, the vital parameter includes a portion of different blood components of blood in a blood vessel of the living being, wherein the portions of the different blood components of the blood influence a wavelength dependence of optical attenuation in the part of the body between the light source and the light receiver. In this case, the sensor is implemented to determine a wavelength dependence of the optical attenuation in the part of the body between the light source and the light receivers.

In another embodiment, the light source and the light receiver are arranged to allow a transmission measurement through the part of the body. Alternatively, the light source and the light receiver may also be arranged to allow a remission measurement through the part of the body.

In another embodiment, the mounting means is implemented to attach the sensor around a human carpus, a human wrist or a human forearm. It has shown that in particular in the fields mentioned vital parameters of the living being can be determined particularly well from the optical attenuation between the light source and the light receiver, without causing unduly great limitation of the freedom to move for the living being and/or for the human being. The movements occurring in the region of the carpus, the wrist or the forearm can be detected by the acceleration sensor so that compensation of the movement artifacts caused by the movement is possible. The mounting means may exemplarily include a rigid or flexible bracelet the size of which is designed to be applied to a human carpus, a human wrist or a human forearm.

In another embodiment, the mounting means is implemented to attach the sensor around a human wrist. In this case, the light source is attached to the mounting means to radiate light into the wrist from an outward side of the wrist. In this case, the light receiver is still attached to the mounting means to receive light from an inward side of the wrist. It has shown that detecting the vital parameters is possible with particularly great advantages when the wrist is radiated through by light from its outward side towards its inward side. In such an assembly of light source and the light receivers, it is ensured that a light propagation through the joint takes place such that the intensity of the light received by the light receiver is maximum. In addition, a sensor worn around the wrist typically is small a limitation for a human patient, the sensor basically corresponding in its wearing qualities to a wrist watch.

Furthermore, it is advantageous for the mounting means, the light source and the light receiver to be implemented to mount the sensor to a part of the body or around a part of a body such that an artery in the part of the body is arranged between the light source and the light receiver. By the arrangement mentioned, it is achieved that the light intensity received by the light receiver has a maximum dependence on the state of the artery, exemplarily on the volume of the artery and the quality of the blood in the artery. Thus, maximum sensitivity of the inventive sensor is ensured.

In another embodiment, the sensor includes, as processing means, pulse determining means implemented to determine a pulse frequency of the living being from temporal variations of the light intensity signal, the pulse frequency of the living being representing the vital parameter. In other words, in an embodiment, the processing means for a combining processing of the light intensity signal and the acceleration signal is attached to the sensor itself or is at least considered to be part thereof. The processing means here is implemented to process and/or combine the light intensity signal and the acceleration signal together to either compensate an influence of the acceleration on the light intensity signal or to generate a combined signal including corresponding information on the light intensity signal and/or the vital parameter and, additionally, on a reliability of the light intensity signal and/or the vital parameter. In other words, the processing means may be implemented to provide a sequence of information pairs, each information pair including information on the vital parameter and associated information on the reliability of the vital parameter, the information on the reliability of the vital parameter being determined based on the acceleration signal.

As an alternative, the processing means may be implemented to only provide the information on the vital parameter when the acceleration signal is in a predetermined allowed range, and to otherwise provide error information.

In another embodiment, the sensor includes a plurality of light sources of different light wavelengths and is implemented to radiate light of different wavelengths into the part of the body. In this case, the sensor is further implemented to determine optical attenuation between the light sources and the light receiver in dependence on the wavelength.

In another embodiment, the sensor includes a plurality of light receivers of different spectral sensitivities and is implemented to allow determining optical attenuation between the light source and the light receivers in dependence on the wavelength. It is possible in both cases to infer a composition of blood in an artery between the light source and the light receiver from the dependence of the optical attenuation on the wavelength of the light.

In another embodiment, the sensor, as processing means, includes blood composition determining means which is implemented to determine portions of different blood components of blood in a blood vessel of the living being from the wavelength dependence of the optical attenuation in the part of the body between the light source and the light receiver. The processing means in this case is implemented to combine or connect the light intensity signal and the acceleration signal to determine the portions of the different blood components in dependence on both the light intensity signal and the acceleration signal. The acceleration signal may then be used for correction or for determining the reliability of certain values, as has already been discussed before.

In an embodiment, the sensor includes the processing means and the processing means is implemented to correct the light intensity signal in dependence on the acceleration signal to counteract changes in the light intensity signal due to the acceleration. Thus, even when there is an acceleration, a reliable light intensity signal describing an actual optical attenuation corrected by the acceleration and/or effects caused by the acceleration in the part of the body between the light source and the light receiver is nevertheless obtained.

In another embodiment, the sensor includes the processing means, the processing means being implemented to determine the information on the vital parameter from the light intensity signal, and the processing means being further implemented to correct the information on the vital parameter in dependence on the acceleration signal to counteract an error of the information on the vital parameter due to the acceleration. In other words, the processing means can receive a light intensity signal influenced by the acceleration and then consider the acceleration signal when calculating the vital parameter from the light intensity signal.

In other words, there are different ways of where in the processing chain the acceleration signal has an effect. Thus, in an embodiment of the present invention, the acceleration signal can be used to correct the light intensity signal and thus to obtain, even when an acceleration acts on the sensor, a light intensity signal corresponding to a light intensity signal without the influence of the acceleration. In another embodiment, with the influence of acceleration, an incorrect light intensity signal is generated, however the influence of the acceleration is eliminated or minimized when determining the information on the vital parameter by the means for deriving the information on the vital parameter from the light intensity signal receiving the acceleration signal and adapting, in dependence on the acceleration signal, the algorithm for determining the information on the vital parameter from the light intensity signal (exemplarily by a change in parameters dependent on the acceleration or by a linear or non-linear combination of signals occurring when determining the information on the vital parameter and the acceleration signal).

In another embodiment, the sensor includes the processing means, the processing means being implemented to determine the information on the vital parameter from the light intensity signal. In the embodiment mentioned, the processing means is also implemented to generate reliability information associated to the information on the vital parameter from the acceleration signal, the reliability information indicating high reliability of the information on the vital parameter with a small-magnitude acceleration and indicating lower a reliability of the information on the vital parameter with a, as far as magnitude is concerned, greater acceleration. Thus, exemplarily information on the vital parameter is calculated independently of the acceleration, however information on the reliability thereof is determined in addition to the information on the vital parameter. This reliability information may, for example, also be considered in further processing of the information on the vital parameter. If, for example, a mean value (exemplarily a temporal mean value) is formed over the information on the vital parameter, the reliability information can be used to perform weighting, i.e. exemplarily to associate a high weight to the information on the vital parameter when forming the weighted mean value when the information on the vital parameter is considered to be reliable due to the reliability information. In contrast, a low weight may be associated to the information on the vital parameter when the information on the vital parameter is considered to be less reliable.

In another embodiment, the sensor includes processing means which is implemented to only determine the information on the vital parameter from the light intensity signal when the acceleration signal indicates that the acceleration is within a predetermined allowable region and to otherwise provide, instead of the information on the vital parameter, information on the vital parameter determined before or an error signal indicating an error, or not to provide information on the vital parameter. In other words, when it is determined that the acceleration is outside the allowable region and thus in this case no reliable information on a vital parameter can be determined, it has proved to be of advantage to exemplarily output again information on the vital parameter determined before. Thus, the information on the vital parameter are output in a continuous sequence, wherein at times where there is a strong acceleration, no update of the information on the vital parameter takes place, but rather a vital parameter determined before is output. This functionality is based on the finding that typically the vital parameter has not changed considerably during the comparatively short time interval during which there is a great acceleration. Furthermore, it has shown that in typical movement patterns of a patient and/or human being or living being there are, with sufficient regularity, states during which the acceleration is sufficiently small so that a vital parameter can be determined reliably with sufficient frequency. In the implementation mentioned, the inventive device provides a continuous sequence of information on the vital parameter, wherein changes of the vital parameter can be recognized sufficiently fast, and wherein thus reliable information on the vital parameter can be output for any point in time. Alternatively, the sensor may also provide an error signal and/or no information on the vital parameter during time intervals in which the acceleration is unduly high. In this manner, an evaluation unit coupled to the sensor can be prevented effectively from receiving unreliable information on the vital parameter.

In another embodiment, the sensor additionally includes attenuation measure detecting means which is implemented to determine attenuation information describing optical attenuation between the light source and the light receiver, and light source adjusting means which is implemented to adjust a light power radiated by the light source in dependence on the attenuation information. In other words, the light source adjusting means receives the optical attenuation between the light source and the light receiver and regulates the light power radiated by the light source into the part of the body such that the light receiver receives a light power sufficient for reliable operation. It is achieved by the inventive detection of the attenuation measure that the intensity of the light source can be adjusted to an optimum value. Thus, it is avoided that the light source radiates too high a light power, which, among other things, would result in an unduly high power consumption and, consequently, an unduly short battery life. On the other hand, it is also avoided that the light source radiates too small a light power, which would result in unreliability of the light intensity signal provided by the light receiver.

In another embodiment, the attenuation measure detecting means is implemented to determine information on a water portion in the tissue of a part of the body and to derive the attenuation information from the information on the water portion. It has shown that the water portion in the tissue has strong influence on the optical attenuation between the light source and the light receiver. Thus, the light intensity is adjusted in dependence on an expected optical attenuation between the light source and the light receiver. It is pointed out here that adjusting the light power emitted by the light source based on the information on the water portion in the tissue, compared to optical regulation (exemplarily based on the light intensity signal as a controlled variable), has the great advantage that no complicated filtering of the light intensity signal is necessary. In particular variations in the light intensity signal represent useful information which of course must not be set to zero. In addition, determining the water portion in the tissue is independent of interfering effects, such as, for example, bones temporarily positioned between the light source and the light receiver. In summary, it can be stated that adjusting the light power radiated by the light source in dependence on the information on the water portion in the tissue ensures particularly reliably adjusting the light intensity influenced by interfering effects, such as, for example, accelerations, bones being present, the volume of blood vessels and the consistency of the blood, to a particularly low degree.

In another embodiment, the attenuation measure detecting means is implemented to determine a skin impedance of the part of the body and to derive the attenuation information from the skin impedance. It has shown that the skin impedance, i.e. an impedance between two electrodes which are in contact with different locations of the skin, provides a reliable statement on the water contents of the tissue and thus the attenuation features of the part of the body.

An embodiment of the present invention further includes processing means for providing information on a vital parameter of a living being based on a light intensity signal and an acceleration signal from a sensor, the light intensity signal describing an intensity of light received by a light receiver attached to the living being from a part of the body of the living being, the acceleration signal describing an acceleration at the location of an acceleration sensor connected to the light receiver. The processing means includes means which is implemented to combine the light intensity signal and the acceleration signal to find out the vital parameter. In other words, the processing means can correct the light intensity signal based on the acceleration sensor, generate combined information including both the light intensity signal and the acceleration signal or prevent the light intensity signal from being generated based on the acceleration signal.

It is also to be pointed out that the processing means can be supplemented by all the features having been described already with regard to the processing means belonging to the sensor.

In addition, an embodiment of the present invention includes a method for providing information on a vital parameter of a living being. The method includes determining information on optical attenuation in a part of the body of the living being between a light source and a light receiver, the optical attenuation depending on the vital parameter, and the light source and the light receiver being attached to the part of the body by mounting means. In addition, the method includes determining information on an acceleration of the light source, the light receiver or the mounting means, and combining the information on the optical attenuation and the information on the acceleration to obtain the information on the vital parameter.

Furthermore, an embodiment of the present invention includes a method for providing information on a vital parameter of a living being in means comprising a light source and a light receiver which are arranged to determine information on optical attenuation in a part of the body of the living being between the light source and the light receiver, the optical attenuation depending on the vital parameter, and the light source and the light receiver being attached to the part of the body by mounting means. The method includes determining information on an acceleration of the light source, the light receiver or the mounting means. Additionally, the method includes switching off the light source and/or adjusting and/or interrupting the generation of the information on the vital parameter, should the information on the acceleration indicate that the acceleration is greater than a predetermined maximum allowable acceleration.

Additionally, an embodiment of the present invention includes a computer program for realizing the inventive method.

### BRIEF DESCRIPTION OF THE DRAWINGS

Embodiments of the present invention will be detailed subsequently referring to the appended drawings, in which:

FIG. 1A is a schematic illustration of an inventive sensor for providing information on a vital parameter of a living being according to a first embodiment of the present invention;

FIG. 1B is a schematic illustration of an inventive sensor for providing a vital parameter of a living being according to a second embodiment of the present invention;

FIG. 2A is a schematic illustration of inventive processing means for a combining processing of a light intensity signal and an acceleration signal according to a third embodiment of the present invention;

FIG. 2B is a schematic illustration of inventive processing means for a combining processing of a light intensity signal and an acceleration signal according to a fourth embodiment of the present invention;

FIG. 2C is a schematic illustration of inventive processing means for a combining processing of a light intensity signal and an acceleration signal according to a fifth embodiment of the present invention;

FIG. 3A is a cross-sectional illustration of an inventive sensor mounted around a human forearm;

FIG. 3B shows an inclined picture of an inventive sensor mounted around a human forearm;

FIG. 4 is a schematic illustration of an inventive sensor including a circuit arrangement for driving the light source and a circuit arrangement for evaluating the light intensity signal;

FIG. 5 is a block circuit diagram of an inventive circuit arrangement setting for adjusting a light quantity emitted by a light source based on skin impedance according to another embodiment of the present invention;

FIG. 6 is a block circuit diagram of an inventive method according to an another embodiment of the present invention; and

FIG. 7 is a block circuit diagram of an inventive method according to another embodiment of the present invention.

### DETAILED DESCRIPTION

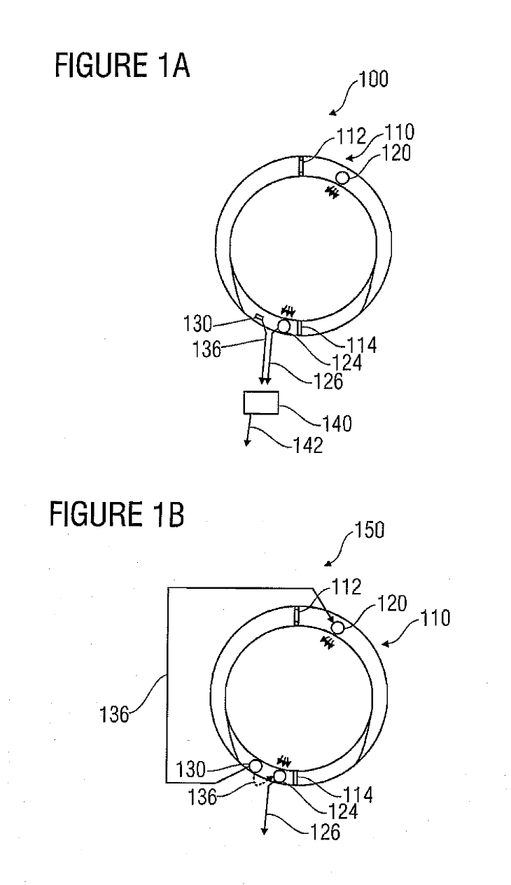

FIG. 1A is a schematic illustration of an inventive sensor for providing information on a vital parameter of a living being according to a first embodiment of the present invention. The sensor according to FIG. 1A in its entirety is referred to by 100. The sensor includes mounting means 110 for attaching the sensor to the living being. The mounting means 110 may, for example, be a bracelet with a hinge 112 and a clasp 114. Alternatively, the mounting means 110 may also be implemented as a bracelet as is exemplarily used with wrist watches. The mounting means 110 may further be produced integrally from an elastic material and may be implemented to be mounted in one piece to a part of the body of the living being. The mounting means 110 may, for example, be made of a metall.

Alternatively, the mounting means 110 may be made of plastic. A light source 120 which is implemented and/or arranged to radiate light into a part of the body of the living being is mounted to the mounting means 110. When exemplarily the mounting means 110 is a bracelet or an arm ring which is implemented to be mounted around a human carpus, a human wrist or a human arm, the light source 120 is arranged to radiate light into the carpus, the wrist or the arm. In other words, when the mounting means 110 exemplarily is an arm ring or a bracelet, the light source 120 is arranged to radiate light towards the inward side of the bracelet or the arm ring.

Very generally, it can be stated that the light source 120 is implemented to radiate light into the part of the body which is at least partly surrounded by the mounting means 110.

In addition, a light receiver 124 is attached to the mounting means 110. The light receiver 124 is implemented to receive a part of the light radiated into the part of the body and to provide a light intensity signal 126 depending on the vital parameter, in dependence on an intensity of the light received. For this purpose, the light receiver 124 is attached to the mounting means 110 such that a direction of maximum sensitivity of the light receiver 124 is oriented towards the inward side of the mounting means 110 and/or towards a part of the body at least partly enclosed by the mounting means 110. When the mounting means 110 is an arm ring or a bracelet, the light receiver 124 is attached to the inward side of the arm ring or bracelet or at least implemented to be able to receive light from the inward side of the arm ring or the bracelet.

In addition, the sensor 100 includes an acceleration sensor 130 connected to the mounting means 110. The acceleration sensor is implemented to provide an acceleration signal 136 in dependence on an acceleration in at least one direction. The acceleration sensor 130 thus is coupled mechanically to the mounting means 110 and basically experiences the same acceleration as the mounting means 110. In addition, the acceleration sensor 130 is coupled mechanically to the light receiver 124 via the mounting means 110 and for this reason experiences the same acceleration as does the light receiver 124 at least when there is an acceleration in a certain direction. In addition, the acceleration sensor 130 is coupled mechanically to the light source 120 so that movements of the light source 120 can typically be detected by an acceleration occurring in the acceleration sensor 130.

Of course, it is to be mentioned here that it is no requirement that exactly the same accelerations must occur at the location of the acceleration sensor 130 as at the location of the light source 120 or the light receiver 124. On the other hand, however, a plurality of movements of the mounting means 110 have at least a similar effect on the light source 120, the light receiver 124 and the acceleration sensor 130 so that, with a plurality of possible movements, the acceleration occurring at the location of the acceleration sensor 130 is a measure of the intensity of a movement of the mounting means 110 and/or the light source 120 and/or the light receiver 124.

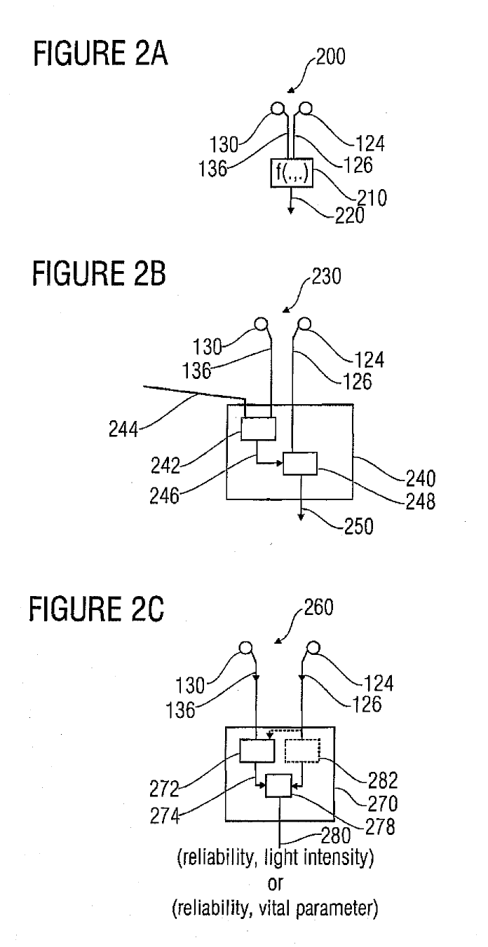

Additionally, the inventive sensor 130 is implemented to transfer the light intensity signal 126 and the acceleration signal 136 to processing means 140 for a combining processing of the light intensity signal 126 and the acceleration signal 136. In other words, the sensor is implemented to transfer the light intensity signal 126 and the acceleration signal 136 in a temporally coordinated manner to single processing means 140. The processing means 140 for a combining processing of the light intensity signal 126 and the acceleration signal 136 may optionally further be part of the sensor 100 and may exemplarily be implemented to provide information 142 on the vital parameter. Details with regard to the potential internal structure of the processing means 140 are exemplarily described referring to FIGS. 2A, 2B and 2C.

The inventive sensor 100 thus allows common and/or combining processing of the light intensity signal 126 and the acceleration signal 136, thereby improving the reliability of the information 142 on the vital parameter generated by the processing means 140, compared to conventional sensors. Influences of the acceleration determined by the acceleration sensor 130 on the light intensity signal 126 or on the information 142 on the vital parameter can be minimized.

It is also to be mentioned here that in an embodiment the light source 120 and the light receiver 124 may be implemented and/or arranged to allow transmission measurement through a part of the body at least partly enclosed by the mounting means 110. In other words, the light source 120 and the light receiver 124 are arranged and/or oriented such that the light source 120 emits a maximum light intensity in the direction towards the light receiver 124 and that the light receiver 124 has a maximum sensitivity in the direction towards the light source 120.

In an alternative embodiment, the light source 120 and the light receiver 124 may also be oriented and/or arranged for remission measurement such that light leaving the light source 120 is scattered and/or reflected in the part of the body towards the light receiver 124.

Furthermore, it is advantageous for the light source 120 and the light receiver 124 to be arranged such that an artery of the living being is between the light source 120 and the light receiver 124 when the mounting means is attached to the living being. The artery may be on a connecting line between the light source 120 and the light receiver 124 or the artery may alternatively be at least in a light path (which may include scattering or reflection) between the light source 120 and the light receiver 124.

FIG. 1B is a schematic illustration of an inventive sensor for providing information on a vital parameter of a living being according to a second embodiment of the present invention. The sensor according to FIG. 1B in its entirety is referred to by 150. Since the sensor 150 is very similar to the sensor 100 according to FIG. 1A, same means and/or signals in the sensors 100, 150 are referred to by the same reference numerals and will not be discussed again.

The sensor 150 includes, as does the sensor 100, a light source 120, a light receiver 124 and an acceleration sensor 130. The acceleration signal 136 provided by the acceleration sensor 130 here is used to generate the light intensity signal 126 in dependence on the acceleration signal 136. In an embodiment of the sensor 150, the acceleration signal 136 exemplarily acts on the light source 120 to switch off the light source 120 should the acceleration signal 136 indicate that an acceleration acting on the sensor 150 is greater than a maximally allowed acceleration. In this case, no light intensity signal 126 is generated or the light intensity signal 126 takes on a minimal value or dark value by deactivating the light source 120. This measure saves energy necessary for operating the light source 120 when the acceleration sensor 136 recognizes that the acceleration acting on the sensor 150 is too great to be able to generate a reliable light intensity signal 126. Alternatively or additionally, the acceleration signal 136 may further be fed to the light receiver 124 to exemplarily deactivate the light receiver 124 when the acceleration signal 136 indicates an acceleration greater than a maximally allowed acceleration. Thus, for example by a direct effect of the acceleration sensor 130 on the light receiver 124, the light receiver 124 is prevented from outputting an unreliable light intensity signal 126 when the acceleration acting on the sensor exceeds a predetermined threshold value.

The acceleration signal 136 may exemplarily have the effect that the light receiver 124 will no longer output a light intensity signal 126 when the acceleration acting on the sensor 150 is too great (greater than a maximally allowed acceleration).

Alternatively, the light receiver may be implemented to continue outputting a predetermined light intensity value when the acceleration signal 136 indicates an unduly great acceleration. Additionally, the light receiver 124 may alternatively be implemented to output an error signal should the acceleration signal 136 indicate an unduly great acceleration.

It is ensured by the measures mentioned that the light intensity signal 126 does not provide invalid values unnoticed, which would result in misinterpretation and/or incorrect measurements in processing means receiving the light intensity signal 126.

FIG. 2A is a schematic illustration of inventive processing means for a combining processing of a light intensity signal and an acceleration signal according to a third embodiment of the present invention. The schematic illustration of FIG. 2A in its entirety is referred to by 200. Processing means 210 is implemented to receive a light intensity signal 126 from a light receiver 124. In addition, the processing means 210 is implemented to receive an acceleration signal 136 from an acceleration sensor 130. The processing means 210 is further implemented to combine the light intensity signal 126 and the acceleration signal 136 according to a predetermined algorithm to obtain as output signal 220 either a light intensity signal corrected by effects due to acceleration or to obtain as output signal information on the vital parameter corrected by effects due to acceleration.

Thus, the acceleration signal may exemplarily be used to adjust and/or set parameters of a signal processing arrangement receiving the light intensity signal 126 for generating the output signal 220 based thereon, in dependence on the acceleration signal 136. In addition, the processing means 210 may be implemented to combine the light intensity signal 126 and the acceleration signal 136 exemplarily by means of addition, subtraction or multiplication. In addition, the processing means 210 may additionally or alternatively be implemented to exemplarily integrate the acceleration signal (exemplarily over time) to obtain information on a speed or a location of the mounting means by a single or double integration of the acceleration signal and to consider the information mentioned when determining the output signal. Correspondingly, the light intensity signal 126 can be combined not only with the acceleration signal 136 itself, but also with a signal resulting from a single or multiple integration of the acceleration signal 136. Furthermore, when calculating the first signal 220, a (continuous or discrete) temporal derivation of the light intensity signal 126 may also be used alternatively or additionally to the light intensity signal 126 itself. Alternatively or additionally, the light intensity signal 126 may also be integrated once or several times (exemplarily over time) to obtain the output signal 220.

FIG. 2B is a schematic illustration of inventive processing means for a combining processing of a light intensity signal and an acceleration signal according to a fourth embodiment of the present invention. The schematic illustration according to FIG. 2B in its entirety is referred to by 230. Processing means 240 receives a light intensity signal 126 from a light receiver 124 and an acceleration signal 136 from an acceleration sensor 130. The processing means 240 includes a reference value comparer 242 comparing the acceleration signal 136 to a maximally allowed acceleration value 244. In other words, the comparing means 242 determines whether the acceleration signal 136 is within an allowed range or not.

The comparing means 242 provides a comparison result 246 to outputting means 248. Should the comparison result 246 indicate that the acceleration is within the allowed range, the outputting means 248 will pass on the light intensity signal 126 as output signal 250 (exemplarily unchanged). If, however, the comparison result 246 indicates that the acceleration is outside the allowed range (exemplarily is unduly great), the outputting means 248 will exemplarily output an error signal as output signal 250. Alternatively, the outputting means 248 may also be implemented to continue outputting a light intensity signal 126 determined before in the case of an unduly great acceleration (exemplarily when the comparison result 246 indicates an unduly great acceleration). In other words, the outputting means 248 may include a port and/or data port and/or latch which passes on a current light intensity signal 126 as long as the comparison result 246 indicates that the acceleration is within the allowed range, and which prevents a change in the output signal 250 should the comparison result 24G indicate that the acceleration is unduly great.

FIG. 2C is a schematic illustration of inventive processing means for a combining processing of a light intensity signal and an acceleration signal according to a fifth embodiment of the present invention. The schematic illustration according to FIG. 2C in its entirety is referred to by 260. Processing means 270 receives a light intensity signal 126 from a light receiver 124 and an acceleration signal 136 from an acceleration sensor 130. The processing means 270 includes reliability determining means 272 which is implemented to generate reliability information 274 based on the acceleration signal 136. The reliability determining means 272 may, for example, be implemented to associate different reliability information 274 to accelerations of different sizes or different types described by the acceleration signal 236. The reliability determining means 272 may be implemented to consider, apart from a magnitude of the acceleration described by the acceleration signal 136, also a direction of the acceleration described by the acceleration signal 136. In other words, the acceleration signal 136 can describe an acceleration in several directions so that the reliability determining means 274 may (also optionally) also evaluate a direction of the acceleration. In addition, the reliability determining means 272 may optionally also evaluate the light intensity signal 126 when determining the reliability information 274.

The reliability determining means 272 may exemplarily be implemented to adjust, with great an acceleration, the reliability information 274 such that it indicates low reliability, and to adjust, with smaller an acceleration, the reliability information 274 such that it indicates greater a reliability. In addition, the reliability determining means 272 may optionally be implemented to adjust, with a light intensity signal 126 having a great magnitude, the reliability information 274 such that it indicates great reliability, and to adjust, with smaller a value of the light intensity signal 126, the reliability information 274 such that it indicates lower a reliability. Additionally, the processing means 270 includes outputting means 278 which is implemented to generate an output signal 280 carrying combined information including both the reliability information 274 and the light intensity signal 126 or information extracted from the light intensity signal 126. Exemplarily, the outputting means 278 may be implemented to output as output signal 280 data pairs including a light intensity derived from the light intensity signal 126 and an associated reliability derived from the reliability information 274.

Alternatively, the processing means 270 may additionally include vital parameter determining means 282 which is implemented to receive the light intensity signal 126 and to provide information on a vital parameter to the outputting means 278 based on the light intensity signal 126. In this case, the outputting means 278 is implemented to provide as output signal 278 a data stream including both information on the vital parameter and associated reliability information. In other words, in this case the output signal includes data pairs including information on a vital parameter and associated information on the reliability of the information on the vital parameter.

In another embodiment, the processing means 270 is implemented to find out by combining the light intensity signal 126 and the acceleration signal 136 whether an information contents of the light intensity signal 126 is plausible. Exemplarily, it may be examined whether changes in the light intensity signal 126 have a temporal correlation with an acceleration occurring. In this case, it can be assumed that the changes in the light intensity signal can be attributed to the acceleration and thus should not be used for an evaluation. Thus, the light intensity signal in this case can be characterized as being unreliable. However, if there is a strong acceleration, but the light intensity signal 226 does not indicate a significant change at the point in time when the acceleration occurs, it may also be assumed that the light intensity signal 126 is reliable in spite of the comparably great acceleration present (stronger than a predetermined acceleration limiting value). Thus, it can be examined by the processing means 270 whether exemplarily there is a temporal coordination between changes in the light intensity signal 126 and a strong acceleration occurring (stronger than a predetermined acceleration limiting value). Only if there is a temporal connection, the light intensity signal 126 can be characterized as being unreliable, whereas in all other cases the light intensity signal 126 is characterized as reliable.

Thus, a plausibility check of the light intensity signal 126 may occur and the light intensity signal 126 is correspondingly exemplarily characterized as unreliable and not passed on to further processing should significant changes (changes greater than a predetermined threshold value) occur in the light intensity signal 126 in a time shortly before or shortly after a strong acceleration occurring (exemplarily within a predetermined time interval around a strong acceleration occurring).

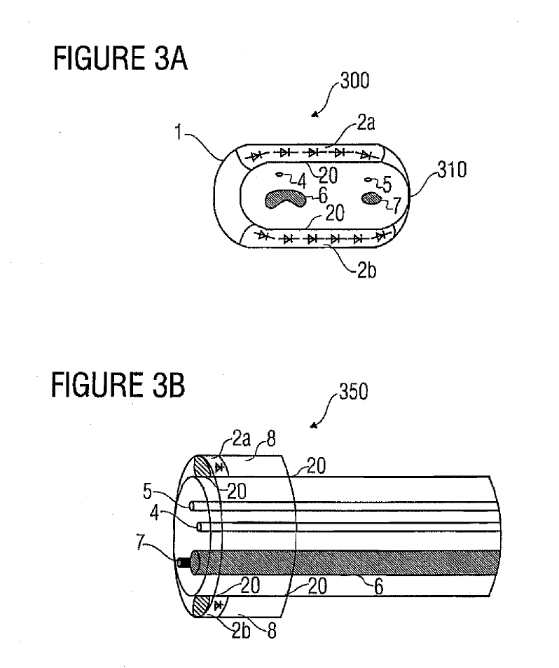

For further explanation, the spatial arrangement of an inventive sensor will be described subsequently referring to FIGS. 3A and 3B, when the sensor is exemplarily attached around a human arm (exemplarily forearm). FIG. 3A is a cross-sectional illustration of an inventive sensor attached around a human forearm. The cross-sectional illustration according to FIG. 3A in its entirety is referred to by 300. A wrist cuff 1 which in the embodiment according to FIG. 3A serves as mounting means encloses a human arm 310 at least partly. A light source matrix 2a serving as light source is attached to the wrist cuff 1. The light source matrix 2a includes at least one light-emitting diode, a plurality of light-emitting diodes, which are implemented to emit light of different spectral compositions. In other words, in an embodiment, a first light-emitting diode of the light source matrix 2a is implemented such that the light generated by the first light-emitting diode comprises an intensity maximum of a first light wavelength λ₁. A second light-emitting diode, however, is implemented such that light emitted from the second light-emitting diode comprises an intensity maximum at a second light wavelength λ₂, the second light wavelength λ₂ differing from the first light wavelength λ₁.

The wrist cuff 1 further includes a photosensitive receiver matrix 2b including at least one light-sensitive diode. But the light-sensitive receiver matrix 2b includes a plurality of light-sensitive diodes. Further, it is advantageous (but not absolutely necessary) for the light-sensitive diodes of the receiver matrix 2b to comprise different spectral sensitivities.

Very generally, it can be stated that it is sufficient for the present invention for the light source matrix 2a to include at least one light-emitting diode (or another light source) and for the photosensitive receiver matrix 2b to include at least one light-sensitive diode (or another light-sensitive element). However, it is advantageous for the light source matrix 2a to include a plurality of light-emitting diodes (or different light sources) and for the photosensitive receiver matrix 2b to include a plurality of light-sensitive diodes (or other photosensitive elements). In addition, it is advantageous (but not absolutely necessary) for the light source matrix 2a to include diodes (and/or other light sources) of different spectral distributions of the light emitted. Additionally, it is avantageous (but not absolutely necessary) for the photosensitive receiver matrix 2b to include light-sensitive diodes and/or photodiodes (or other light-sensitive elements) of different spectral sensitivities. In order to determine a spectral form of optical attenuation between the light source matrix 2a and the photosensitive receiver matrix 2b, it is sufficient for either the light source matrix 2a to comprise light-emitting diodes of different spectral distributions or for the photosensitive receiver matrix 2b to comprise light-sensitive diodes of different spectral sensitivities.

In addition, it is pointed out that the forearm 310 includes a bone 6 called radius and a bone 7 called ulna. In addition, the forearm 310 includes a radius artery 4 called arteria radialis and an ulna artery 5 called arteria ulnaris. The radius artery 4, the ulna artery 5, the radius 6 and the ulna 7 are arranged in the forearm 310 in the manner known from medicine.

The light source matrix 2a (also abbreviated as light source) and the photosensitive receiver matrix 2b (also abbreviated as light receiver) are arranged at the wrist cuff 1 (also referred to as mounting means) such that at least one artery (exemplarily the radius artery 4 or the ulna artery 5) is between a light-emitting diode (or generally a light source) of the light source matrix 2a and a light-sensitive diode (or generally a light-sensitive element) of the photosensitive receiver matrix 2b when the wrist cuff 1 is mounted to a human forearm or around a human wrist.

In addition, two electrodes or skin electrodes 20 are arranged at the wrist cuff 1 such that the skin electrodes 20 are in electrically conductive connection with the skin of the forearm 310 or the wrist or the carpus when the wrist cuff 1 is attached to the forearm 310, the wrist or the carpus. The skin electrodes 20 are further coupled to means for impedance measurement in order to determine an impedance between the skin electrodes 20, as will be explained in greater detail below.

FIG. 3B additionally shows an inclined image of an inventive sensor mounted around a human forearm. The graphical illustration of FIG. 3B in its entirety is referred to by 350. Since the graphical illustration 350 only differs from the graphical illustration 300 by the perspective chosen, same means and/or features have the same reference numerals in graphical illustrations 300 and 350. Thus, a repeated explanation thereof is omitted.

However, it is pointed out that exemplarily the light source matrix 2a is attached to the wrist cuff 1 such that the light source matrix 2a is adjacent to the inward side of the forearm, the inward side of the wrist or the inward side of the carpus when the wrist cuff 1 is attached to the forearm 310, around the wrist or around the carpus. Additionally, it is advantageous for the photosensitive receiver matrix 2b to be arranged at the wrist cuff 1 such that the light-sensitive receiver matrix 2b is adjacent to an outward side of the forearm, the wrist or the carpus when the wrist cuff 1 is attached to the forearm, around the wrist or around the carpus.

Alternatively, the light source matrix 2a may also be attached to the wrist cuff 1 such that the light source matrix 2a is adjacent to the outward side of the forearm 310, the outward side of the wrist or the outward side of the carpus when the wrist cuff 1 is attached to the forearm, around the wrist or around the carpus. In this case, the photosensitive receiver matrix 2b is arranged at the wrist cuff 1 such that the photosensitive receiver matrix 2b is adjacent to the inward side of the forearm, the inward side of the wrist or the inward side of the carpus when the wrist cuff 1 is attached to the forearm, around the wrist or around the carpus.

In addition, it is advantageous for the wrist cuff 1 to be implemented such that the wrist cuff 1 is fixed around the wrist when the wrist cuff is attached to the wrist, that the wrist cuff 1 thus is not shiftable in the direction of the carpus or in the direction of the forearm when the wrist cuff is attached around the wrist. Thus, it is ensured that the measurement will be at the optimum position, namely in direct proximity to the wrist.

It becomes obvious from FIG. 3B that additionally an acceleration sensor 8 is attached to the wrist cuff 1. Thus, different positions may be chosen for the acceleration sensor. In an embodiment, the acceleration sensor 8 is arranged adjacent to the light source matrix 2a so that the acceleration sensor 8 and the light source matrix 2a are on the same side (inward side or outward side) of the forearm, the wrist or the carpus. Thus, it is exemplarily ensured that the acceleration sensor records an acceleration acting on the light source matrix 2a. It has been recognized that a shift of the light source matrix 2a relative to the forearm, the wrist or the carpus has particularly strong an influence on the light intensity signal provided by the photosensitive receiver matrix 2b.

In another embodiment, the acceleration sensor 8 is arranged adjacent to the photosensitive receiver matrix 2b so that the acceleration sensor 8 is on the same side (inward side or outward side) of the forearm, the wrist or the carpus as the photosensitive receiver matrix. Such an arrangement is also of particular advantage since a great error may form in the light intensity signal when the photosensitive receiver matrix 2b is shifted relative to the forearm, the wrist or the carpus by an acceleration.

In another embodiment, two or more acceleration sensors may be arranged at different positions of the wrist cuff 1, exemplarily both adjacent to the light source matrix 2a and adjacent to the photosensitive receiver matrix 2b.

The signals of the two or more acceleration sensors may then be combined or may be used to write and/or detect accelerations in different directions.

In other words, the present invention according to an embodiment provides a photoplethysmograph based on the transmission principle wearable at the wrist. The plethysmograph in one example includes a matrix-shaped arrangement consisting of several blocks of several light sources of different wavelengths which is exemplarily formed by the light source matrix 2a. In addition, the plethysmograph includes, according to an embodiment, a matrix-shaped arrangement of photosensitive elements consisting of several blocks the spectrum of which (and/or spectral sensitivity) is tuned to the wavelengths used (exemplarily at the light sources). The matrix-shaped arrangement of photosensitive elements is in one embodiment formed by the photosensitive receiver matrix 2b.

In one embodiment, the photoplethysmograph additionally includes an acceleration sensor for each of three axes (or directions) in space. Alternatively, the photoplethysmograph may also include only one or two acceleration sensors for one direction or for two directions. The acceleration sensors (or the acceleration sensor) serve for improving signal quality and provide a measure for evaluating a plausibility of a plethysmogram recorded.

The usage of light sources of different wavelengths according to an embodiment allows adjusting the photoplethysmograph to a skin color and to an anatomy of the wrist and further allows drawing conclusions to blood components (exemplarily blood in an artery between the light source matrix 2a and the photosensitive receiver matrix 2b).

According to another embodiment, the casings supported on the skin surface (exemplarily the forearm, the wrist or the carpus) and containing the light sources and/or the photosensitive elements (or the casing containing the light sources and the photosensitive elements) are designed such that the casings (or the casing) may be used as at least two skin electrodes 20 for measuring skin impedance.

According to an embodiment, a water portion in tissue is inferred to from the skin impedance.

Exemplarily, a degree of exsiccation of a patient, as well as blood viscosity and the risk of stroke connected thereto can optionally be derived from the water portion in the tissue.

Thus, the present invention is based on the concept of detecting a plethysmogram at the wrist (of, for example, a human being or a living being) by arranging a light source 120, 2a of suitable wavelength with suitable driving at an outward side of the wrist opposite a photosensitive element 124, 2b on the inward side of the wrist such that at least one of the arm arteries (arteria radialis 4 or arteria ulnaris 5) is between the light source 120, 2a and the photosensitive element 124, 2b.

According to another aspect, the invention is based on the concept that, by means of measuring of the skin impedance, the water portion in the tissue can be derived and, from this, the degree of exsiccation and blood viscosity and a risk of stroke connected thereto.

Another central idea of the present invention is that blood components may be inferred from a suitable driving method of the light source 120 and/or the light source matrix 2a and the light receiver 124 and/or photosensitive receiver matrix 2b, using different wavelengths.

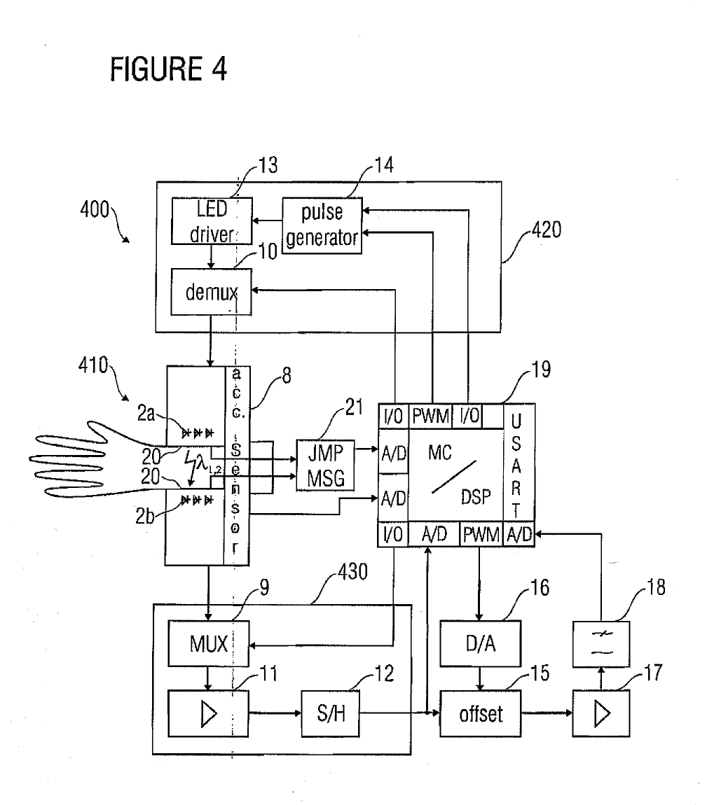

FIG. 4 is a schematic illustration of an inventive sensor including a circuit assembly for driving the light source and for evaluating the light intensity signal. The arrangement according to FIG. 4 in its entirety is referred to by 400.

The core of the arrangement 400 is a measuring receiver 410 exemplarily including a wrist cuff 1, a light source matrix 2a, a photosensitive receiver matrix 2b, an acceleration sensor 8 and optionally at least two skin electrodes 20, as has exemplarily been described referring to FIGS. 1A, 1B, 3A and 3B.

In an embodiment, the light source matrix 2a and the photosensitive receiver matrix 2b are implemented (but not necessary so) to use at least two light wavelengths λ₁, λ₂. Alternatively, only one light wavelength λ may be used in a simple embodiment.

The circuit arrangement 400 is controlled by a microcontroller and/or a digital signal processor 19 exemplarily providing a plurality of digital output lines and further implemented, for itself or in combination with further peripherals, to read in several analog signals. Controlling the circuit arrangement 400 may alternatively be performed by a discrete analog and/or digital circuit.

The circuit arrangement 400 additionally includes a driving unit 420 implemented to drive the one or several light-emitting diodes of the light source matrix 2a. The driving circuit 420 includes a pulse generator 14 implemented to generate impulses for driving an LED driver 13. The LED driver 13, in connection with the pulse generator 14, makes available voltage impulses or current impulses serving to drive the light-emitting diode of the light source matrix 2a. Should the light source matrix 2a include more than one diode, a demultiplexer 2a will distribute the voltage impulses or current impulses generated by the LED driver 13 to the light-emitting diodes of the light source matrix 2a. Exemplarily, the demultiplexer may be implemented to pass on a voltage impulse or current impulse provided by the LED driver 13 to precisely one selected light-emitting diode from a plurality of light-emitting diodes or to precisely one selected group of light-emitting diodes from a plurality of groups of light-emitting diodes of the light source matrix 2a. The demultiplexer 10, among other things, receives selection information from the microcontroller or digital signal processor 19 determining which light-emitting diode or group of light-emitting diodes is to be excited by a voltage impulse or current impulse. In an embodiment, the pulse generator 14 is also driven by the microcontroller or the digital signal processor 19, thereby exemplarily determining an impulse duration and/or an impulse intensity of the voltage pulse or current pulse passed on to the light-emitting diodes.

The circuit arrangement 400 further includes receiving means 430 coupled to the photosensitive receiver matrix 2b and implemented to evaluate the voltage signals or current signals provided by the photosensitive receiver matrix. In an embodiment, the receiver circuit 430 includes a multiplexer 9 implemented to select a signal from a light-sensitive diode of the photosensitive receiver matrix 2b (or from a group of light-sensitive diodes of the photosensitive receiver matrix 2b) for being passed on to an amplifier 11. Thus, the multiplexer 9 is driven by the microcontroller or digital signal processor 19. In addition, the receiver circuit 430 includes a sample and hold circuit 12 coupled to the output of the amplifier 11 and thus implemented to sample and hold a signal provided by a light-sensitive diode selected by the multiplexer 9 and amplified by the amplifier 11. The output of the sample and hold circuit 12 is further coupled to an analog input of the microcontroller or digital signal processor 19, wherein the signal provided by the sample and hold circuit 12 is converted to a digital signal.

As an alternative, an external analog-to-digital converter which is coupled to the microcontroller or digital signal processor 19 may of course also be employed.

The output signal of the sample and hold circuit 12 is in an embodiment further fed to an offset circuit 15. The offset circuit 15 is implemented to shift the output signal of the sample and hold circuit 12 by an offset, i.e. exemplarily to reduce or eliminate a direct portion in the offset signal. The offset circuit 15 exemplarily receives a signal from a digital-to-analog converter 16 driven by the microcontroller or digital signal processor 19. Exemplarily, the microcontroller or digital signal processor 19 includes means for pulse width modulation (PWM) to allow the signal for the offset circuit 15 to be provided. In this case, the digital-to-analog converter 16 may exemplarily only include a low-pass filter to convert the pulse width modulated signal provided by the pulse width modulation circuit to a corresponding direct voltage. However, a conventional analog-to-digital converter exemplarily receiving a digital signal from the microcontroller or digital signal processor and making available based thereon an input signal for the offset circuit 15 may be used as an alternative. In other words, the offset circuit 15 exemplarily forms a difference between the output signal of the sample and hold circuit 12 and the signal provided by the digital-to-analog converter circuit 16. The result of forming the difference, i.e. the output signal of the offset circuit 15, is fed to a series connection of an amplifier 17 and a low-pass filter 18. It is achieved by means of the circuit arrangement mentioned that the amplifier only receives an alternating portion of the output signal of the sample and hold circuit 12 and that thus the alternating portion of the output signal of the sample and hold circuit is amplified and filtered. The output signal provided by the filter 18 is fed to an analog-to-digital conversion, wherein the microcontroller or digital signal processor 19 may include an analog-to-digital converter and be coupled to such an analog-to-digital converter to convert the output signal of the filter 18 to a digital signal.

The circuit arrangement 400 thus is implemented to determine optical attenuation between the light source matrix 2a and the photosensitive receiver matrix 2b for at least one light wavelength and for at least one pair of light sources (exemplarily at least one light-emitting diode) and light receivers (exemplarily at least one light-sensitive diode). By using a demultiplexer circuit 10 and a multiplexer circuit 9, using a simple hardware, the attenuation between the light source matrix 2a and the photosensitive receiver matrix 2b may, among other things, be determined for a plurality of light wavelengths λ and/or for a plurality of geometrical propagation paths.

The circuit arrangement 400 further includes an acceleration sensor 8 mechanically coupled to the light source matrix 2a and/or the photosensitive receiver matrix 2b. Thus, the acceleration sensor provides information describing the acceleration acting on the light source matrix 2a or on the photosensitive receiver matrix 2b. The microcontroller or digital signal processor 19 typically receives the information on the acceleration, as an analog signal and is implemented to convert the information on the acceleration to a digital signal and to consider it, when evaluating the information provided by the photosensitive receiver matrix 2b, as has already been explained in greater detail before.

The microcontroller or digital signal processor 19 additionally includes a universal serial, synchronous or asynchronous transmitter and/or receiver (USART) for communicating with further components of a system. It is to be pointed out that the microcontroller or digital signal processor 19 is typically implemented or programmed to provide information on a vital parameter of the human being carrying the inventive sensor based on the information provided by the photosensitive receiver matrix 2b. As an alternative, the microcontroller or digital signal processor 19 may also determine and/or provide intermediate information from which the vital parameter and/or the information on the vital parameter may be derived.

The sensor 410 further (optionally) includes two skin electrodes 20 arranged at the sensor 410 to be in contact with the skin of a living being wearing the sensor 410. A circuit arrangement 21 is coupled to the skin electrodes 20 to perform an impedance measurement of an impedance between the skin electrodes 20. The circuit arrangement 21 for measuring an impedance also provides information on the impedance at the microcontroller or digital signal processor 19. The circuit arrangement 21 for measuring an impedance provides an analog signal fed to an analog input of the microcontroller or digital signal processor 19.

The evaluation and/or usage of the mentioned information on the impedance between the skin electrodes 20 will be described below in greater detail.

In summary, it can be stated that a suitable microcontroller, exemplarily a digital signal processor 19, performs driving the individual components of the arrangement 400 and recording, processing and evaluating the signal forms resulting from the arrangement 400.

Thus, the circuit arrangement 400 includes a pulse generator 14 generating suitable voltage forms for driving the LED driver 13. The demultiplexer 10 performs distributing the signals generated to the individual light sources 2a arranged in a matrix and distributed in blocks. The signals of the acceleration sensors 8 (and/or an acceleration sensor 8) are digitalized and processed by the microcontroller or digital signal processor 19. A circuit 21 receives the signals of the skin electrodes 20 and feeds same in a suitable manner to the microcontroller or digital signal processor digitalizing and processing the signals of the skin electrodes. The multiplexer 9 provides for receiving and passing on the signals from the individual light-sensitive elements 2b arranged in a matrix and distributed to blocks to the sample hold element 12. Downstream of the sample hold element 12, the signal is digitalized and processed by the microcontroller or digital signal processor 19. The signal of the sample hold element 12 is fed to an offset circuit 15 driven by the microcontroller or digital signal processor 19 via the digital-to-analog converter 16. Subsequently, the signal is amplified by a circuit 17 and filtered by a circuit 18. After that, the signal is digitalized and processed by the microcontroller or digital signal processor 19.

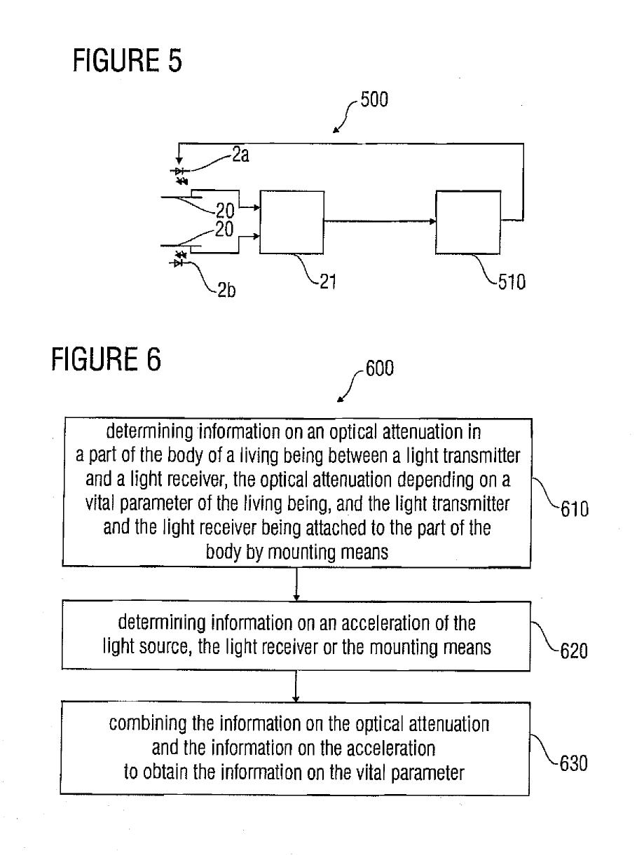

FIG. 5 shows a block circuit diagram of an inventive arrangement for adjusting a light quantity emitted by a light source based on a measurement of the skin impedance.

The circuit arrangement according to FIG. 5 in its entirety is referred to by 500. The circuit arrangement 500 is suitable for being used in an inventive sensor for determining a vital parameter of the living being. Decisive for the applicability of the circuit arrangement 500 is the fact that a sensor for determining a vital parameter includes a light source 2a (exemplarily in the form of a single light source and/or light-emitting diode or in the form of a light source matrix) and a light receiver 2b (exemplarily in the form of a single light receiver and/or a single light-sensitive diode or in the form of a photosensitive receiver matrix), wherein tissue of a part of the body is radiated through by light emitted by the light source 2a to be received and/or detected by the light receiver 2b. In addition, the light source 2a and the light receiver 2b are typically attached to mounting means which in turn is implemented to being attached to the part of the body. The mounting means carrying the light source 2a or the light receiver 2b additionally includes two skin electrodes 20 connected to the mounting means to be in electrically conductive contact with a skin surface of the part of the body enclosed by the mounting means when the mounting means is attached to the part of the body.

The skin electrodes 20 are additionally integrated in the casing of the mounting means carrying and/or housing the light source 2a and/or the light receiver 2b. In other words, the skin electrodes 20 exemplarily form part of a surface of the mounting means casing.

An impedance measuring circuit 21 is coupled to the skin electrodes 20 in an electrical (or electrically conductive) fashion and is implemented to determine an impedance between the skin electrodes 20. The impedance determining circuit 21 may exemplarily be implemented to determine only a real part of an impedance between the skin electrodes 20 and only determine an imaginary part of an impedance between the skin electrodes 20 or determine both a real part and an imaginary part of an impedance between the skin electrodes 20. It has shown that the impedance between the skin electrodes 20 exemplarily is a measure of a water portion in a tissue arranged between the skin electrodes 20 and that in addition the measure of the water contents in the tissue describes optical attenuation when light passes from the light source 2a to the light receiver 2b.

In other words, the impedance determining means 21 very generally allows, in connection with the skin electrodes 20, determining (and/or estimating) an optical attenuation between the light source 2a and the light receiver 2b, the optical attenuation being determined in a non-optical way. In other words, the attenuation is determined by measuring an electrical characteristic of the part of the body. Alternatively, optical measurement of the optical attenuation is also possible.

The circuit arrangement 500 further includes light quantity adjusting means 510 coupled to the impedance measuring means 21 to receive information on a skin impedance between the electrodes 20 from the impedance measuring means 21. Furthermore, the light quantity adjusting means 510 is implemented to act on the driving of the light source 2a (and/or to drive the light source 2a) to adjust the light energy or light power emitted by the light source 2a in dependence on the information provided by the impedance determining means 21. In an embodiment, the light quantity adjusting means 510 is implemented to adjust the light energy and/or light power radiated by the light source 2a into the part of the body to great a value when the impedance between the skin electrodes 20 has small a value, and to adjust the light energy or light power radiated into the part of the body to a comparatively lower value when the impedance between the skin electrodes 20 takes on a comparatively greater value. In an alternative embodiment, the light quantity adjusting means 510 is implemented to radiate the light power radiated by the light source 2a into the part of the body such that the light power accepts greater a value when there is greater an impedance between the two skin electrodes 20 than when there is a comparatively smaller impedance between the skin electrodes 20.

In another embodiment, the light quantity adjusting means is implemented to derive, based on the information, provided by the impedance determining means 21, on the impedance between the electrodes 20, information on a water portion in the tissue of the part of the body between the skin electrodes 20 and to adjust, based on the information on the water portion in the tissue, the light energy or light power radiated by the light source 2a into the part of the body.

In another embodiment, the light quantity adjusting means 510 is implemented to determine, based on the information on the water portion in the tissue of the part of the body between the skin electrodes 20, information on an optical attenuation in the tissue of the part of the body between the skin electrodes 20 and to derive the light energy or light power radiated by the light source 2a into the part of the body in dependence on the information on the optical attenuation in the tissue of the part of the body.

In other words, the light energy or light power radiated by the light source 2a into the part of the body may be determined in a multi-stage process in which two or more steps may be summarized to form a single step. The potential individual steps include: determining the impedance between two skin electrodes which are in electrical (and/or electrically conductive) contact with the part of the body; determining a water portion in the tissue of the part of the body based on the information on the impedance between the skin electrodes; determining information on an optical attenuation in the part of the body based on the information on the water portion in the tissue of the part of the body; and adjusting the light energy or light power based on the information on the optical attenuation in the part of the body.

Determining the information on the water portion in the part of the body and determining the information on the optical attenuation in the part of the body may optionally be omitted, i.e. adjusting the light energy or light power of the light source 2a may take place directly based on the impedance between the skin electrodes. In other words, a real part of the impedance, an imaginary part of the impedance, a magnitude of the impedance or a phase of the impedance may exemplarily be mapped by the light quantity adjusting means 510 to a light energy or light power of the light source 2a. The mapping may, for example, take place by a functional connection or using a table of values, wherein a light energy or light power is associated each to a certain impedance (such as, for example, a real part, an imaginary part, a magnitude or a phase of the impedance). FIG. 6 shows a flow chart of an inventive method for providing information on a vital parameter of a living being.

The method according to FIG. 6 in its entirety is referred to by 600. The method 600 includes determining 610 information on an optical attenuation in a part of the body of the living being between a light transmitter and a light receiver, the optical attenuation depending on a vital parameter of the living being. The light transmitter and the light receiver are attached to the part of the body by mounting means.

Additionally, the method includes, in a second step 620, determining information on an acceleration of the light source, the light receiver or the mounting means.

Furthermore, the method 600 includes, in a third step 630, combining the information on the optical attenuation and the information on the acceleration to obtain information on the vital parameter.

Furthermore, it is to be pointed out that the method 600 may be supplemented by all those steps performed by the inventive device described above.

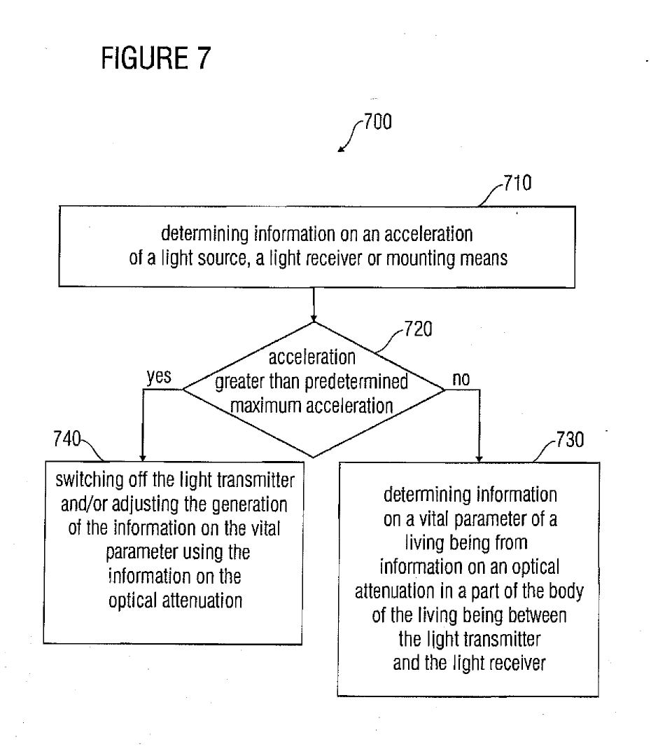

FIG. 7 shows a flow chart of an inventive method for providing information on a vital parameter of a living being in means comprising a light source and a light receiver which are arranged to determine information on an optical attenuation in a part of the body of the living being between the light source and the light receiver, the optical attenuation depending on the vital parameter, and the light source and the light receiver being attached to the part of the body by mounting means. The method according to FIG. 7 in its entirety is referred to by 700. In a first step 710, the method 700 includes determining information on an acceleration of the light source, the light receiver or the mounting means. In a second step 720, an examination is performed whether the acceleration is greater or smaller than a predetermined maximum acceleration. If the acceleration is smaller and/or not greater than the predetermined maximum acceleration, in step 730, information on the vital parameter of the living being is determined from information on an optical attenuation in the part of the body of the living being between the light transmitter and the light receiver. If, however, it is determined in step 720 that the acceleration is greater than the predetermined maximum acceleration, the light transmitter will be switched off and/or the generation of the information on the vital parameter using the information on the optical attenuation will be stopped and/or interrupted.

The method 700 may additionally be supplemented by all those steps performed by the inventive device described above.

The inventive method may be realized in any way. Exemplarily, the inventive method may be realized by electronic computing equipment and/or by a computer.

In other words, the inventive device and the inventive method may be implemented in either hardware or in software. The implementation may be on a digital storage medium, exemplarily on a disc, CD, DVD, ROM, PROM, EPROM, EEPROM or FLASH memory having control signals which may be read out electronically, which can cooperate with a programmable computer system such that the corresponding method will be executed.

In general, the present invention thus also is in a computer program product comprising a program code stored on a machine-readable carrier for performing at least one of the inventive methods when the computer program product runs on a computer. In other words, the invention may also be realized as a computer program having a program code for performing an inventive method when the computer program runs on a computer.

In summary, it can be stated that the present invention includes a sensor device for non-invasively recording a plethysmogram at the wrist of a human being. According to one aspect, the present invention includes simultaneous measurement of the movement of the sensor device and the skin impedance. A plethysmogram here is a graphical representation of volume changes, such as, for example, of arteries of a living being.

Among other things, the present invention is based on the finding that photoplethysmographs (exemplarily photoplethysmographs for being used at the finger which are exemplarily attached to the fingertip by a finger clip) are highly reactive to vibration, making reliable recording and/or evaluation of the plethysmogram very difficult.

Thus, it is the object of the present invention to detect a plethysmogram at the wrist by a sensor device based on the transmission principle. The sensor device is worn at the wrist to reduce a limitation of the freedom to move for a human being to a minimum. Acceleration sensors, exemplarily for the coordinate axes in a three-dimensional space, detect movements and vibrations of the sensor device and allow post-correction and plausibility evaluation of a plethysmogram maybe affected by movement artifacts. A skin impedance at the wrist is measured by means of electrodes and the water portion in a tissue which considerably contributes to attenuating the light radiated is determined therefrom. Corresponding to the water portion in the tissue, the light power (and/or light energy) radiated (into the tissue) is adjusted optimally. Thus, a reduction in the energy consumption (relative to conventional plethysmographs in which a predetermined light power and/or light energy is used) is achieved. Furthermore, (optionally) a degree of exsiccation and a blood viscosity of the patient are determined from the water portion (in the tissue) to estimate the risk of stroke.

The inventive plethysmograph is worn at the wrist, similarly to a wrist watch, and not at the finger of a patient (as has conventionally be the case). Thus, the finger is not blocked and the patient is not limited in his or her freedom to move. The inventively used motional sensors (and/or acceleration sensors) allow plausibility evaluation and correction of a plethysmogram which may be affected by movement artifacts. The plethysmogram is recorded more reliably since the plethysmograph is insensitive towards vibrations. By measuring the skin impedance, the degree of exsiccation may be inferred from the water portion of the tissue and the risk of stroke can be estimated. In addition, in the inventive manner, the light power radiated can be adjusted optimally and the energy consumption of the sensor device can be minimized.

Thus, the present invention generally provides a device for determining a vital parameter, insensitive to movements and/or vibrations, based on a light intensity signal generated by radiating through a part of the body of a patient using a light source and a light receiver attached to the patient using mounting means. Considering movements, more precise results can be achieved than using conventional measuring means, and at the same time a maximum freedom to move for a patient and/or living being may also be ensured wile measurements are performed.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations, and equivalents as fall within the true spirit and scope of the present invention.

1. A sensor for providing information on a vital parameter of a living being, comprising:
a mounter for attaching the sensor to the living being;
a light source connected to the mounter for radiating light into a part of the body of the living being;
a light receiver connected to the mounter and implemented to receive part of the light radiated to provide, in dependence on an intensity of the light received, a light intensity signal depending on the vital parameter; and
an acceleration sensor connected to the mounter and implemented to provide an acceleration signal in dependence on an acceleration in at least one direction,
wherein the sensor is implemented to transfer the light intensity signal and the acceleration signal to a processor for a combining processing of the light intensity signal and the acceleration signal or to generate the light intensity signal in dependence on the acceleration signal.

2. The sensor according to claim 1, wherein the vital parameter includes a pulse frequency of a living being, and wherein the light source and the light receiver are arranged such that an optical attenuation in the part of the body between the light source and the light receiver is influenced by a change in volume in a blood vessel in the part of the body.

3. The sensor according to claim 1, wherein the vital parameter includes a portion of different blood components of blood in a blood vessel of the living being, wherein the portions of the different blood components of the blood influence a wavelength dependence of an optical attenuation in the part of the body between the light source and the light receiver, and
wherein the sensor is implemented to determine the wavelength dependence of the optical attenuation in the part of the body between the light source and the light receiver.

4. The sensor according to claim 1, wherein the light source and the light receiver are arranged to allow transmission measurement through the part of the body.

5. The sensor according to claim 1, wherein the mounter is implemented to attach the sensor around a human carpus, a human wrist or a human forearm.

6. The sensor according to claim 5, wherein the mounter is implemented to attach the sensor to a human wrist, and wherein the light source is attached to the mounter to radiate light into the wrist from an outward side of the wrist.

7. The sensor according to claim 5, wherein the mounter is implemented to attach the sensor around a human wrist, and wherein the light receiver is attached to the mounter to receive light from an inward side of the wrist.

8. The sensor according to claim 1, wherein the mounter is implemented to attach the sensor around a human ankle bone, a human ankle joint or a human lower leg.

9. The sensor according to claim 1, wherein the mounters, the light source and the light receiver are implemented to mount the sensor to a part of the body or around a part of the body such that an artery in the part of the body is arranged between the light source and the light receiver.

10. The sensor according to claim 1, wherein the sensor includes, as a processor, a pulse determiner implemented to determine a pulse frequency of the living being from temporal variations of the light intensity signal, the pulse frequency of the living being representing the vital parameter.

11. The sensor according to claim 1, wherein the sensor includes a plurality of light sources of different light wavelengths and is implemented to radiate light of different wavelengths into the part of the body to allow determining an optical attenuation between the light sources and the light receiver in dependence on the wavelength.

12. The sensor according to claim 1, wherein the sensor includes a plurality of light receivers of different spectral sensitivities to allow determining an optical attenuation between the light source and the light receivers in dependence on the wavelength.

13. The sensor according to claim 1, wherein the sensor includes, as a processor, a blood composition determiner implemented to determine, using a wavelength dependence of the optical attenuation in the part of the body between the light source and the light receiver, portions of different blood components of blood in a blood vessel of the living being.

14. The sensor according to claim 1, wherein the sensor includes the processor, and wherein the processor is implemented to correct the light intensity signal in dependence on the acceleration signal to counteract changes in the light intensity signal due to the acceleration.

15. The sensor according to claim 1, wherein the sensor includes the processor, wherein the processor is implemented to determine the information on the vital parameter from the light intensity signal, and wherein the processor is additionally implemented to correct the information on the vital parameter in dependence on the acceleration signal to counteract a change in the light intensity signal due to the acceleration.

16. The sensor according to claim 1, wherein the sensor includes the processor,
wherein the processor is implemented to determine the information on the vital parameter from the light intensity signal and to generate from the acceleration signal reliability information associated to the information on the vital parameter which indicates high reliability of the information on the vital parameter with small an acceleration magnitude and indicates lower a reliability of the information on the vital parameter with greater an acceleration magnitude.

17. The sensor according to claim 1, wherein the sensor includes the processor, and wherein the processor is implemented to only determine the information on the vital parameter from the light intensity signal or output same if the acceleration signal indicates that the acceleration is within a predetermined allowed region, and to otherwise provide, instead of current information on the vital parameter, information, determined before, on the vital parameter or an error signal indicating an error.

18. The sensor according to claim 1, wherein the sensor includes the processor, wherein the processor is implemented to only determine the information on the vital parameter from the light intensity signal if the acceleration signal indicates that the acceleration is within a predetermined allowed region, and otherwise not to provide information on the vital parameter.

19. The sensor according to claim 1, the sensor further comprising:
an attenuation measure detector implemented to determine attenuation information describing an optical attenuation between the light source and the light receiver; and
a light source adjuster implemented to determine a light power or light energy radiated by the light source in dependence on the attenuation information.

20. The sensor according to claim 19, wherein the attenuation measure detector is implemented to determine information on a water portion in a tissue of the part of the body and to derive the attenuation information from the information on the water portion.

21. The sensor according to claim 19, wherein the attenuation measure detector is implemented to determine a skin impedance of the part of the body and to derive the attenuation information from the skin impedance.

22. The sensor according to claim 19, wherein the mounter includes a first electrode and a second electrode which are arranged to electrically contact the part of the body, the attenuation measure detector being implemented to determine an impedance between the first electrode and the second electrode and to derive the attenuation information from the impedance.

23. The sensor according to claim 1, wherein the sensor includes a light source driver implemented to switch off the light source when the acceleration signal indicates that the acceleration is greater than a maximally allowed acceleration.

24. A processor for providing information on a vital parameter of a living being based on a light intensity signal and an acceleration signal from a sensor, the light intensity signal describing an intensity of light received from a light receiver attached to a living being from a part of the body of the living being, and the acceleration signal describing an acceleration at the location of an acceleration sensor mechanically connected to the light receiver, comprising:
a combiner implemented to combine the light intensity signal and the acceleration signal to determine the information on the vital parameter.

25. A method for providing information on a vital parameter of a living being, comprising:
determining information on an optical attenuation in a part of the body of the living being between a light source and a light receiver, wherein the optical attenuation depends on the vital parameter, and wherein the light source and the light receiver are attached to the part of the body by mounter;
determining information on an acceleration of the light source, the light receiver or the mounter; and
combining the information on the optical attenuation and the information on the acceleration to obtain the information on the vital parameter.

26. A method for providing information on a vital parameter of a living being in connection with a device comprising a light source and a light receiver which are arranged to determine information on an optical attenuation in a part of the body of the living being between the light transmitter and the light receiver, the optical attenuation depending on the vital parameter, and the light source and the light receiver being attached to the part of the body by a mounter, comprising:
determining information on an acceleration of the light source, the light receiver or the mounter; and
should the information on the acceleration indicate that the acceleration is greater than a predetermined maximally allowed acceleration, switching off the light source and/or interrupting a generation of the information on the vital parameter using the information on the optical attenuation;
otherwise determining the information on the vital parameter of the living being from the information on the optical attenuation in the part of the body of the living being between the light source and the light receiver.

27. A computer program for performing a method for providing information on a vital parameter of a living being, comprising: determining information on an optical attenuation in a part of the body of the living being between a light source and a light receiver, wherein the optical attenuation depends on the vital parameter, and wherein the light source and the light receiver are attached to the part of the body by mounter; determining information on an acceleration of the light source, the light receiver or the mounter; and combining the information on the optical attenuation and the information on the acceleration to obtain the information on the vital parameter, when the computer program runs on a computer.

28. A computer program for performing a method for providing information on a vital parameter of a living being in connection with a device comprising a light source and a light receiver which are arranged to determine information on an optical attenuation in a part of the body of the living being between the light transmitter and the light receiver, the optical attenuation depending on the vital parameter, and the light source and the light receiver being attached to the part of the body by a mounter, comprising: determining information on an acceleration of the light source, the light receiver or the mounter; and should the information on the acceleration indicate that the acceleration is greater than a predetermined maximally allowed acceleration, switching off the light source and/or interrupting a generation of the information on the vital parameter using the information on the optical attenuation; otherwise determining the information on the vital parameter of the living being from the information on the optical attenuation in the part of the body of the living being between the light source and the light receiver, when the computer program runs on a computer.
